(19) United States
(12) Patent Application Publication
(10) Pub. No.: US 2024/0266072 A1
(43) Pub. Date: Aug. 8, 2024

(54) SYSTEM AND METHOD FOR GENOMIC ANALYSIS OF PATHOGENS

(71) Applicant: Battelle Memorial Institute, Columbus, OH (US)

(72) Inventors: Craig M. Bartling, Columbus, OH (US); Brett R. Fowle, Columbus, OH (US); Omar P. Tabbaa, Columbus, OH (US)

(21) Appl. No.: 18/563,060

(22) PCT Filed: May 21, 2022
(86) PCT No.: PCT/US22/30410

§ 371 Date: Nov. 21, 2023

Related U.S. Application Data
(60) Provisional application No. 63/191,904, filed on May 21, 2021.

(51) Int. Cl. G16H 50/80; G16B 30/10

(52) U.S. Cl. CPC G16H 50/80; G16B 30/10

(57) ABSTRACT

The present invention is directed towards a bioinformatic method for screening complex patient-derived samples for predicting and identifying at least one pathological agent, especially human pathological agents, comprising establishing agent confidence value from a set of collected patient-derived samples.

### PRIORITY CLAIM

This application claims the priority benefit of U.S. Provisional Patent Application Ser. No. 63/191,904 filed 21 May 2021.

### FIELD OF THE INVENTION

This application is directed to the field of bioinformatics and use of advanced systems and methods to identify and characterize pathological agents obtained from patient derived or other biological samples.

### BACKGROUND OF THE INVENTION

Sequencing DNA, RNA or mRNA means determining the order of chemical building blocks that make up the molecule under test. A sequence may disclose aspects of genetic information that are highly useful for a wide variety of applications (e.g., biosurveillance, diagnostics, and research). Databases and computer-based tools are essential in bioinformatics because of the sheer complexity of analyzing a genome, even for a simple virus. For example, viral genomes may be astoundingly diverse with respect to size, complexity and type of nucleic acid. Viral nucleic acids may be DNA or RNA, double or single stranded, monopartite or multipartite, short (˜2 kb) or long (˜2500 kb). More complex life forms such as mammals will have significantly more complex genomes; the human genome, for example, consists of approximately 3 billion base pairs in the double-helix DNA, residing in the 23 pairs of chromosomes within the nucleus of human cells.

With such enormous amount of detailed data to analyze, high throughput sequencing (“HTS”) is becoming more routine as a tool in bioinformatics. Properly applied, HTS has the potential to help achieve accurate and efficient sample characterization, such as summarization of taxonomic and functional compositions. Although HTS holds promise for increasing knowledge for many applications, the extremely large amount of recovered data requires well-considered analysis methods. Current generations of these bioinformatic methods often can be bottlenecks for successful implementation.

For example, the COVID-19 pandemic has confirmed the reality of both pandemic risk and gaps in preparedness [1]. Current surveillance programs enable monitoring of known-diseases caused by respiratory and enteric viruses, antimicrobial resistant bacteria, and other dangerous pathogens [2-5]. HTS, however, offers some significant advantages over these targeted programs. For one thing, HTS can enable unbiased and enhanced surveillance, promoting one of the goals of surveillance e.g., “to facilitate rapid and appropriate response to outbreaks and virus zoonotic spillover events” [6]. Most critically, in HTS-based active surveillance the pathogen does not have to be known prior to the outbreak. Similar to biosurveillance efforts, clinical metagenomics using HTS is shifting the way physicians are diagnosing and treating diseases [22]. Major advances in computational biotechnologies and supporting infrastructure that enables efficient and accurate screening of large-scale genomic data for the identification of biological threats help to leverage biologically inspired algorithms and highly curated databases of genomes, proteins, and specific genetic determinants of pathogenicity [7-11]. Particularly with infectious diseases, properly analyzed HTS significantly helps diagnosis of sepsis, respiratory diseases, and other infectious diseases [22]. Further yet, biological research routinely uses HTS to characterize a wide range of samples, because accurate taxonomic composition and functional characterization is critical to making sound conclusions.

Thus, for any use cases that leverage HTS data, accurate and efficient bioinformatic methods are needed for accurate taxonomic composition and functional characterization. Critical also is the read, which may be a DNA sequence from a small section of a larger sequence, or an inferred sequence of base pairs, or base pair probabilities. What is needed are systems and methods for the determination of accurate taxonomic compositions from HTS and other genomic datasets.

One of the most important applications of accurate and efficient bioinformatic methods is to predict one or more disease outbreaks. According to the World Health Organization, a disease outbreak is the occurrence of disease cases in excess of normal expectancy. The number of cases varies according to the disease-causing agent, and the size and type of previous and existing exposure to the agent. Disease outbreaks are usually caused by an infection, transmitted through person-to-person contact, animal-to-person contact, or from the environment or other media. Outbreaks may also occur following exposure to chemicals or to radioactive materials. There is a need for improved methods to predict disease outbreaks that are reliable and repeatable.

### SUMMARY OF THE INVENTION

The present invention describes a method for identifying the taxonomic composition of a genomic sequence dataset, and in particular, for identifying or predicting at least one pathological agent in a clinically relevant genomic dataset.

In one aspect, the invention provides a method to identify or to predict a pathological agent, comprising: a) receiving a dataset of metagenomes from uninfected people or nonhuman animals; b) receiving an infected dataset; c) developing a database of clinically relevant proteomes by processing the dataset of metagenomes and the infected dataset; d) aligning datasets using an aligner tool; e) removing alignments with less than 99% identity and an alignment length of less than 48 bps; f) scoring the retained alignments; g) calculating an agent confidence value from the scoring; h) predicting an agent based on a confidence threshold; and i) calculating a performance metric relative to data from a pre-existing method of predicting a disease outbreak.

In another aspect, the invention provides a method for making a taxonomic composition prediction, comprising: a) an alignment step against a subject database of protein and/or nucleotide sequences; b) a filtering step comprising removing low quality alignments; c) a scoring step comprising: scoring sequence level taxonomy predictions based on an information content; combining sequence-level taxonomy prediction scores into sample-level taxonomy scores; d) a finding step comprising finding all reads associated with the highest scoring taxonomy that has not yet been processed and removing all other taxonomies and associated accession from the reads; and wherein the scoring step results in one or more sample-level taxonomy scores; and conducting a K-means cluster of the sample-level taxonomy scores by domain and set the taxonomies associated with a top cluster to a final taxonomic composition prediction. In some embodiments, the invention can be further characterized by one or any combination of the following: step b) includes anomalous reads; wherein the sequence-level taxonomy prediction scoring in step c) is achieved by S_{a,r,acc} produced from:

$${S}_{a , r , acc} = \frac{{Rcov}_{acc , r} \times {Nuacc}_{a}^{n}}{{Na}_{r}^{m} \times {Nacc}_{r}^{p}} ,$$

where Rcov_{acc,r} is a measure alignment quality of the query subsequence associated with region r to subject accession acc, Nuaccₐⁿ is the number of unique accession associated with an agent found across the entire metagenomic sample, Naᵣ is the number of unique agents associated with region r, Naccᵣ is the number of accessions associated with region r, and m, n, and p belong to the integers; wherein the sequence-level taxonomy prediction scoring in step c) is achieved by S_{a,r,acc} produced from:

$${S}_{a , r , acc} = \frac{{Rcov}_{acc , r}}{{Na}_{r} \times {Nacc}_{r}}$$

wherein Rcov_{acc,r} is a measure alignment quality of the query subsequence associated with region r to subject accession acc, Naᵣ is the number of unique agents associated with the subject accessions in the region r, and Naccᵣ is the number of accessions from the subject database that associates with the region; wherein in step c) the sample-level taxonomy scores are achieved by: obtaining a taxonomic composition region score, S_{a,r}, which is calculated to be the score associated with the highest scoring accession for the given taxonomic composition, region combination:

$${S}_{a , r} = {max}_{acc} {S}_{a , r , acc} ;$$

obtaining sample-level taxonomy score, Sₐ, which is calculated as follows:
   Sₐ=ΣᵣS_{a,r}; wherein the method further includes iteratively repeating scoring until all taxonomies have been processed; wherein the alignment database comprises a set of sequences or concern, proteomes from pathogenic sequences, and/or sequences from domains comprises at least two, at least three, or all four of bacteria, archea, eukaryote, and virus; wherein step b) comprises a minimum alignment length of 48 base pairs, and/or 99% nucleotide identity, 100% region coverage, and/or 95% protein identity; wherein step b) comprises removing reads that share the same protein accession; wherein step b) comprises a default abundance threshold of 6% of reads that share the same accession that are associated with high-quality alignments of the sample prior to scoring; wherein the method further comprises assigning the at least one identified pathological agent to a patient, and the patient or that the patient's doctor is informed of the result.

The present invention also provides a method of detecting a disease outbreak, comprising: establishing a cohort of individuals for repeated collection of samples; repeatedly collecting samples from the individuals in the cohort; analyzing the samples to provide identification of at least one pathological agent; and determining whether the at least one identified pathological agent indicates a disease outbreak.

Embodiments described herein further provide a method of making a taxonomic composition prediction, comprising: an alignment step against a subject database of protein and/or nucleotide sequences; a filtering step comprising removing low quality alignments and, optionally, anomalous reads; a scoring step comprising: scoring sequence level taxonomy predictions based on an information content; combining sequence-level taxonomy prediction scores into sample-level taxonomy scores; finding all reads associated with the highest scoring taxonomy that has not yet been processed and removing all other taxonomies and associated accession from these reads; and, optionally, iteratively repeating scoring until all taxonomies have been processed; wherein the scoring step results in one or more sample-level taxonomy scores; and (optionally) conducting a K-means cluster of the sample-level taxonomy scores by domain and setting the taxonomies associated with a top cluster within a particular threshold to a final sample composition prediction.

Further embodiments may be characterized by one or any combination of the following: wherein the cohort comprises consenting patients such as front line workers, dialysis patients, other immunocompromised patients, or another selected cohort; wherein the samples are sequenced using high throughput screening; wherein the identification of at least one pathological agent is conducted; wherein the identified at least one pathological agent is assigned to a patient and the patient or patient's doctor is informed of the result; wherein an alert is transmitted of the indication of a disease outbreak.

Further, the methods described herein may also be characterized by one or any combination of the following: wherein the alignment database comprises a set of sequences or concern, proteomes from pathogenic sequences, and/or sequences from domains comprises at least two, at least three, or all four of bacteria, archea, eukaryote, and virus; wherein the filtering step comprises a minimum alignment length of 48 base pairs, and/or 99% nucleotide identity and 100% region coverage, and/or 90% protein identity; wherein the filtering step removes reads that share the same protein accession (high throughput sequencing results in unbiased sequence amplification; thus, abundant reads that are associated with the exact same protein accession should be removed) and comprise a set percentage (a default abundance threshold of 6% of reads that share the same accession that are associated with high-quality alignments) of the sample prior to scoring.

The present invention further provides a method of proving the effectiveness of a method predicting a disease outbreak, comprising: providing a dataset of metagenomes from uninfected people or nonhuman animals; providing an infected dataset; optionally converting FastQ and fna datasets into Fasta files; providing or developing an alignment database; aligning datasets, preferably using a lambda aligner; filtering out alignments with less than 99% identity and an alignment length of less than 48 bps; calculating agent confidence; predicting agent based on confidence threshold; and calculating a performance metric relative to known data.

It should be understood that in various aspects, embodiments described herein may include any of the detailed methods and/or method steps in whole or in part, including, for example, aspects of detecting pathogens from one or more human samples. In various embodiments, any of the calculations and/or data analysis described herein may be employed in whole or in part for any of the embodiments.

Reference will now be made in detail to various embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, systems, and components have not been described in detail so as not to unnecessarily obscure aspects of the various embodiments. In some instances, the concepts herein may obviate in whole or in part one or more of the problems encountered in the prior art.

### BRIEF DESCRIPTION OF DRAWINGS

FIG. 1 illustrates a sample calculation for calculating agent confidence.

FIG. 2 illustrates Performance of an embodiment of the present inventive bioinformatic method (“PanGuard”), where (A) is using the full proteomes database and agent confidence scoring discussed in the Calculating Agent Confidence section, (B) is using the 45 proteomes from the Karius dataset [15] [See Identifying Clinically Relevant Pathogens and Building the Associated Proteomes Database Section Below] and agent confidence scoring discussed in the organism confidence section, (C) is using the 45 proteomes from the Karius dataset without deduping at the region, accession-level, and (D) is using a selected sequence of concern database (virulence factor database) and only considering unique regions.

FIG. 3 illustrates Performance of PanGuard's bioinformatic platform using the full proteomes databases for spike-in agent copy number where (A) is less 50 and (B) is greater than or equal to 50.

FIG. 4 illustrates Performance of PanGuard's bioinformatic platform for exemplary parameterizations. Performance is quantified by precision (Panel 4A) and recall (Panel 4B) relative to the state of the art (Karius).

FIG. 5 illustrates Performance of PanGuard's bioinformatic platform for E-value cut off's ranging from 1E-10 to 100. Performance is quantified by precision (Panel 5A) and recall (Panel 5B) relative to the state of the art (Karius).

### DETAILED DESCRIPTION OF THE INVENTION

In one embodiment of the present inventive method (PanGuard), FastQ and fna datasets may be converted into Fasta files. Typically, a FastQ file contains the raw data output from a sequencing operation. Each entry may contain four lines comprising a Sequence identifier, the actual sequence, a quality score identifier line (usually consisting only of a ‘+’) and a quality score. A file with a .fna extension stores DNA information typically just nucleic acid information without other DNA-related information such as those with other extensions for example .fa, .ffn, .faa, .frn, .mpfa, .seq .net or .aa. A FASTA data format is a text-based format for representing either nucleotide sequences or amino acid/protein sequences. FASTA is widely used due to its relative simplicity, making the data easy to manipulate and parse using text-processing tools such as scripting languages.

For datasets in FNA format, the method initially removes non-canonical base pairs (bps). These are base pairs that may include those that do not encode amino acids.

The method next develops a database of clinically relevant proteomes, a set of relevant proteins produced in a biological context. The method uses a taxonomy based on the union of organisms covered by Battelle's Sequence of Concern (“SoC”) databases and those covered by the Dx company Karius [15] as more particularly described below.

In an exemplary embodiment, the method then aligns the datasets using a lambda aligner [18] to both Battelle's SoC database and the database of clinically relevant pathogen proteomes. An exemplary alignment is /path/to/lambda2 searchp -q /path/to/query.fa -i /path/to/index.lambda -o /path/to/output.m8 -e 1e-04 [or 100]-n 1000 --output-columns “qseqid sseqid qstart qend sstart send pident qlen slen”. Similar aligners may also be used in other embodiments.

The next step is to remove alignments with less than 99% identity (a tunable parameter) and an alignment length less than 48 bps (set based on aligner amino acid seed length).

Then, the method in at least one embodiment is to calculate agent confidence from the retained alignments, further described below.

Finally, the method in at least one embodiment may predict pathological agent based on the developed confidence threshold.

Generating True Keys for Metagenomic Samples from Literature

In various embodiments, the PanGuard method is applicable to identifying the taxonomic composition of any genomic dataset. As an example, in various embodiments PanGuard may be used to diagnose sepsis from cell free DNA in blood. One option to assess the precision of PanGuard is to compare the PanGuard against the state of the art (SoA) by obtaining “true keys” based on the available data in the literature [14-16, 19-21]. A true key is a file that is compiled to check the results (and calculate precision and recall) of PanGuard. It is based on published papers that have microbiological data for diagnosis (blood culture, etc.). The true key was used as a comparison to PanGuard's analysis, which in various embodiments uses DNA sequence data as an input. When available, both classical clinical Dx test results (e.g., culture, qPCR) were included in addition to the results of the authors. While true keys were compiled for several references [14-16, 19, 21] only the true key derived from Blauwkamp et al. [15] is shown here as it was the basis for comparison described herein.

Table 1 is a summary of an exemplary study and sample, in this case a Sepsis validation study for Karius that included the indicated plasma samples.

TABLE 1

| | # | Microbiology | Karius |
|---|---|---|---|
| Sample Type | Samples | truth? | results? |
| DNA spike: Serum spiked with | 362 | Yes, based on spike | No |
| sheared DNA (Note: 9 of these | | | |
| are negatives—no spike) | | | |
| CMV qPCR samples (note | 25 | Yes, CMV qPCR | No |
| some of these CMV negative) | | results | |
| Asymptomatic patients | 170 | No tests run | No |
| Septic patients: definite* | 117 | Yes, blood culture, | Yes |
| | | qPCR, etc | |
| Septic: probable* | 52 | No | Yes |
| Septic: possible* | 29 | No | Yes |
| Septic: unlikely* | 32 | No | Yes |
| Other (no truth provided) | 118 | No | No |

Within Table 1's first column, “Definite*” means one of the Karius results appeared to be consistent with microbiology results. Other possible terms include “probable*”—this term means the result was not available within seven days of Next Generation Sequencing sample collection, but the sequencing pathogen result was considered the likely cause of sepsis based on clinical, radiological, or laboratory findings. The term “possible*” means sequencing pathogen result had potential for pathogenicity consistent with clinical presentation but an alternative explanation for the symptoms was more likely. Finally, the term “unlikely*” means sequencing result adjudicated as unlikely causes of sepsis if the organism(s) identified had a potential for pathogenicity but were inconsistent with clinical presentation. In other embodiments, additional terms or varying definitions may be useful.

Only results for positive samples are described, as comparing true negatives is not practical and often not possible. While negatives could be compared using the asymptomatic patients, positives were still reported in many of these cases (although the results were not broken down by sample). Thus, the focus of the comparisons in this study is on the “definite” septic patients shown in Table 1.

### Developing a Subject Database for Alignment

In an exemplary embodiment, the methods and systems described here may develop alignment results for any database. Described now is an exemplary method to create an alignment database of clinically relevant pathogen proteomes. More particularly, an exemplary method to generate a list of agents and corresponding proteome IDs comprises the following steps:
1—Generate a list of biological agents included in Battelle's SoC database;
2—Remove non-pathogenic agents and agents that are not likely to impact humans;
3—Combine the list above with at least one reference list of pathogens, such as for example the Karius list of pathogens https://kariusdx.com/pathogenlist/3.6;
4—Deduplicate the combined list;
5—Download all reference proteomes from the Universal Protein Resource (Uniprot) (https://www.uniprot.org/proteomes/?query=&sort=score) or other similar service;
6—Filter out phage proteomes and redundant proteomes to remove Uniparc entries;
7—Compile a list of priority reference proteomes, filtering by Reference proteomes for example better annotated proteomes;
8—Use a fuzzy matching script to annotate each agent with its best matching proteome ID with preference given to reference proteomes;
   From the above proteomes list, two proteome alignment databases may be generated in an exemplary embodiment:
First, a “45 Proteome” database, consisting of proteomes for the 45 pathogens confirmed by microbiology to be in the Karius dataset; and
Second, a “1,000 Proteome” database, consisting of all agents with deference proteomes from UniProt plus proteomes from the 45 Proteome Database that did not have a reference proteome.
   NOTE: ˜300 of the agents could not be matched to proteomes and were not included in the above lists.
   Additionally, the “SoC Database” was also used as an alignment database for comparison. This database contains ˜10,000 sequences (virulence factors, toxins, etc.) from pathogens.

### Calculating Agent Confidence

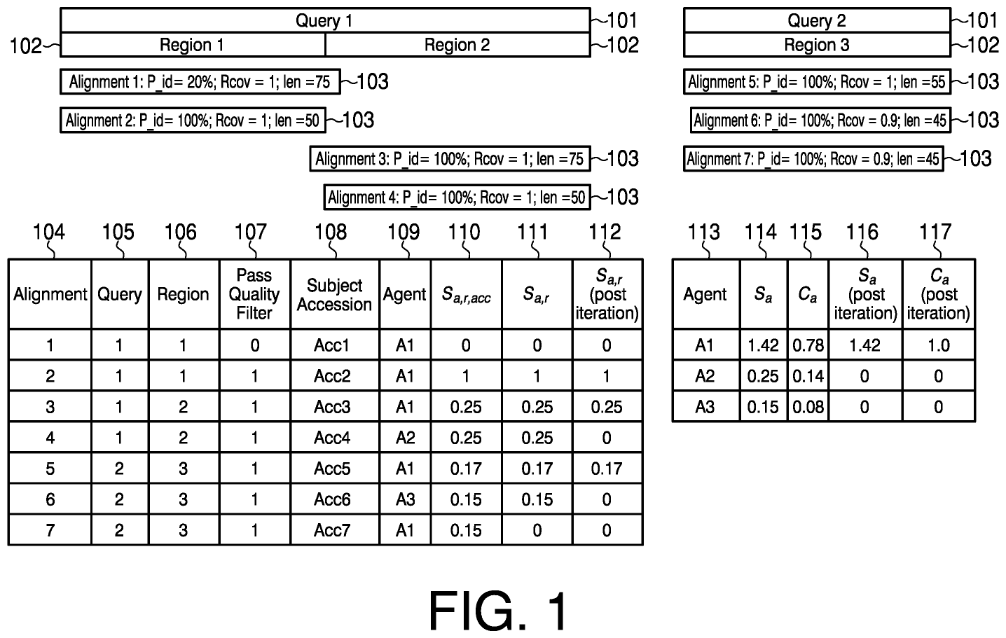

In an exemplary embodiment, agent confidences may be calculated after filtering out alignments that do not pass a percent identity and length threshold denoted in the technical approach. A unique set of queries that pass the indicated thresholds may be collected from tabular alignment results from a sample. Looping through each unique query, regions may be built out of any query starts and ends across the entire length of the query. It should be noted that a region can only be derived from alignments from a single query. For example, given two alignments that overlap from the same query, a region may be defined by the range from the minimum query start to the maximum query end between the overlapping alignments. Alternatively, if region already exists for this query and overlaps with the alignments, the region bounds will be adjusted to the minimum query start and the maximum query end between the overlapping region and alignments. On the other hand, if any alignments are not overlapped with either other alignments or existing regions, new regions may be created for these alignments bounded by their query start and end positions. The following section is illustrated in FIG. 1, a sample calculation for calculating agent confidence. Exemplary original sequence data query results are shown as Queries 101 and exemplary regions shown as regions 102.

In an embodiment, once all regions are compiled scoring initially may be performed on a per region, per agent, and per accession basis. This is shown in exemplary compilations 103. More specifically, each region (r) 106, agent (a) 109, 113, and accession (acc) 108 combination may be assigned a score, S_{a,r,acc} 110:

$${S}_{a , r , acc} = \frac{{Rcov}_{acc , r}}{{Na}_{r} \times {Nacc}_{r}}$$

Where Rcov_{acc,r} is the alignment percent coverage of a subject accession acc 108 in a region r 106. For example, a region may be 100 amino acids and the subject accession may align to 20 amino acids in the region, therefore Rcov_{acc,r}=20%. Naᵣ is the number of unique agents associated with the subject accessions in the region r. Accordingly, score S_{a,r,acc} is inversely proportional to the region's uniqueness to the region's agent specificity. Naccᵣ is the number of accessions from the subject database that may be associated with the region. The score S_{a,r,acc} is inversely proportional to the region's sequence complexity. In other words, higher complexity implies more specificity to a specific protein.

Alternatively, a more general scoring function may be in the form of:

$${S}_{a , r , acc} = \frac{{Rcov}_{acc , r} \times {Nuacc}_{a}^{n}}{{Na}_{r}^{m} \times {Nacc}_{r}^{p}} ,$$

where Rcov_{acc,r} is a measure alignment quality of the query subsequence associated with region r to subject accession acc, Nuaccₐⁿ is the number of unique accession associated with an agent found across the entire metagenomic sample—this parameter is a measure of the coverage of an agent's proteome in the sample (thought to be correlated with increased accuracy in predicting sample composition), Naᵣ is the number of unique agents associated with region r, Naccᵣ is the number of accessions associated with region r, and m, n, and p belong to the integers.
Subsequently, an agent region score, S_{a,r} 111, is calculated to be the score associated with the highest scoring accession (or more than one accession, in the case of a tie) for the given agent, region combination:

$${S}_{a , r} = {max}_{acc} {S}_{a , r , acc}$$

Subsequently, the agent score, Sₐ 114, is calculated as follows:

$${S}_{a} = {\sum }_{r} {S}_{a , r}$$

and the agent confidence, Cₐ 115, is defined as:

$${C}_{a} = \frac{{S}_{a}}{{\sum }_{a} {S}_{a}} .$$

However, in an embodiment, calculated agent confidence may result in values that are overconfident for agents with weaker evidence. For example, if Agent A has a confidence score of 0.5, Agent B has a confidence score of 0.4, and Agent C has a confidence score of 0.1, there is an inference suggesting that Agents A and B may be in the sample. However, after closer inspection it may be discerned that all of Agent B's confidence come from reads associated with Agent A, which can be the case for agents that are taxonomically close relatives. In such a case, agents A and C are likely the only true agents in the sample, as there is no unique evidence pointing to Agent B's existence in the sample and one would expect that Agent A's confidence to remain at 0.5, whereas Agent C's confidence would increase to 0.5, which likely represents the true composition of the sample. Generalizing this issue, in various embodiments, not accounting for such cases similar to this scoring example may increase substantially PanGuard's false positive and false negative predictions. To address this issue, in an embodiment, the final agent confidence scores may be further reviewed and improved, via the following exemplary iterative process:
1. Sort the agents from highest to lowest agent confidence;
2. Pull all regions that are associated with the highest confidence agent;
3. Set the agent, region score equal to zero for all other agents in these regions;
4. Recalculate agent confidences;
5. Pull all regions associated with the next highest confidence agent;
6. Set the agent, region score equal to zero for all other agents in these regions; and
7. Recalculate agent confidences.

Then, repeat steps 5-7 until all remaining agents have been iterated over. Other aspects of FIG. 1 include whether alignments 104 are filtered 107 if they do not pass a percent identity.

Optionally, a K-means clustering of the agent scores by domain can be calculated and the final taxonomies are those associated with the top cluster within each domain (within a threshold). Such a step may be performed to remove spurious results.

### Calculating Precision and Recall

In an embodiment, the method executes a curation process for the set of agents microbiology confirmed to be present in each sample in the Blauwkamp (Karius) dataset, along with the set of agents predicted by Karius to be in one or more samples. One skilled in the art will appreciate that curation can occur only where data is available; Blauwkamp et al. did not appear to report on the majority of the samples. Yet further, it does not appear that any false negatives are reported. Additionally, the set of agents predicted by PanGuard to be in the sample is acquired through Battelle's bioinformatic alignment pipeline and the methods described in at least as per this embodiment. For each sample, the number of true positive, true negative, false positive, and false negative agents may be calculated by comparing the set of predicted agents to the set of verified agents. By iterating through the entire dataset for each set of predictions, the total number of true positives (TP), true negatives (TN), false positives (FP), and false negatives (FN) can be calculated. Using these values, precision and recall are calculated as follows:

$$Precision = \frac{TP}{TP + FP} and Recall = \frac{TP}{TP + FN} .$$

### Results for Pathogen Identification and Characterization

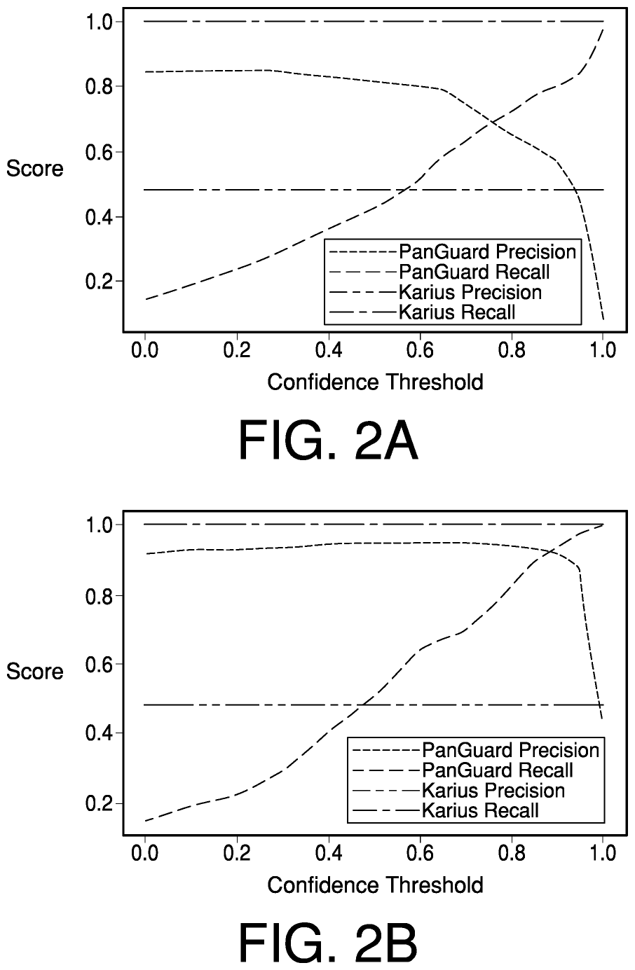

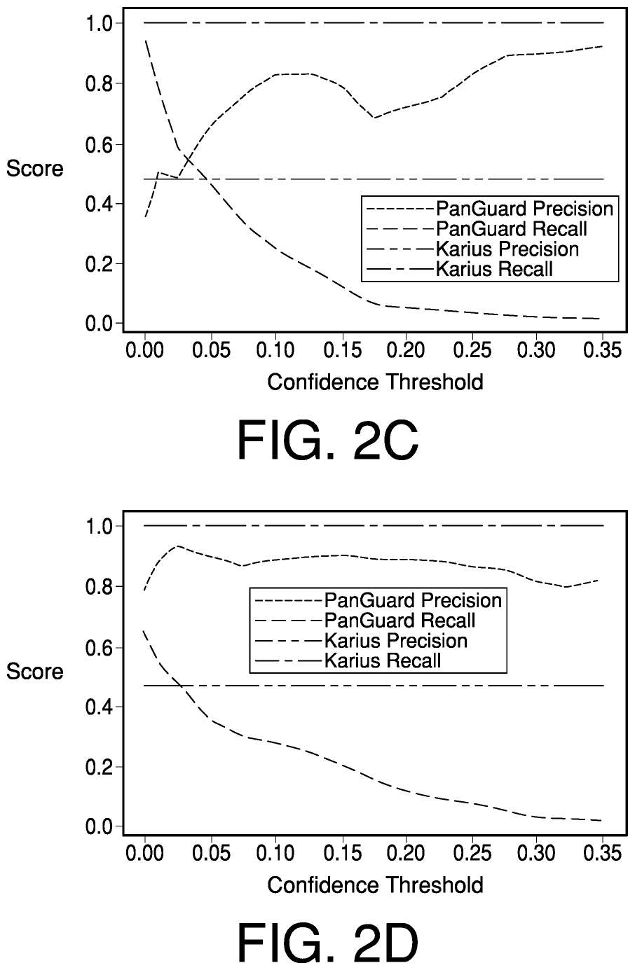

A bioinformatic study was performed to guide the PanGuard bioinformatic platform's system design decisions, as well as compare its expected performance to the state of the art. PanGuard predicted agents are based on the fraction of the maximum agent confidence value for the sample (e.g. FIG. 2 x-axis). More particularly, in an embodiment, for a given sample all agents with confidence greater than X % of the max confidence are selected as predictions. The only exception is for S. epidermidis, as it is a common contaminant in many samples. When S. epidermidis is the top agent, the max confidence threshold is set to the confidence of the second highest confidence agent. FIG. 2 shows the performance of PanGuard's bioinformatic platform under the following conditions. Panel 2A is using the 1,000 proteomes database and agent confidence scoring discussed in the Calculating Agent Confidence section. Panel 2B is using the 45 proteomes from pathogens found in the Karius dataset only considering unique regions. Panel 2C is using the 45 proteomes from the Karius dataset without deduping at the region, accession level. Panel 2D is using the SoC database and only considering unique regions. As shown in the figure, by using the SoC database without filtering unique regions yields similar results to those shown in FIG. 2D, and hence demonstrating the limited redundancy in Battelle curated database.

Panels 2C and 2D show results thresholding on the raw confidence score. While the trends are different in these plots, the performance shown is reflective of the tool under these conditions. Blauwkamp et al. did not report a single false negative, so recall is 1 in all cases.

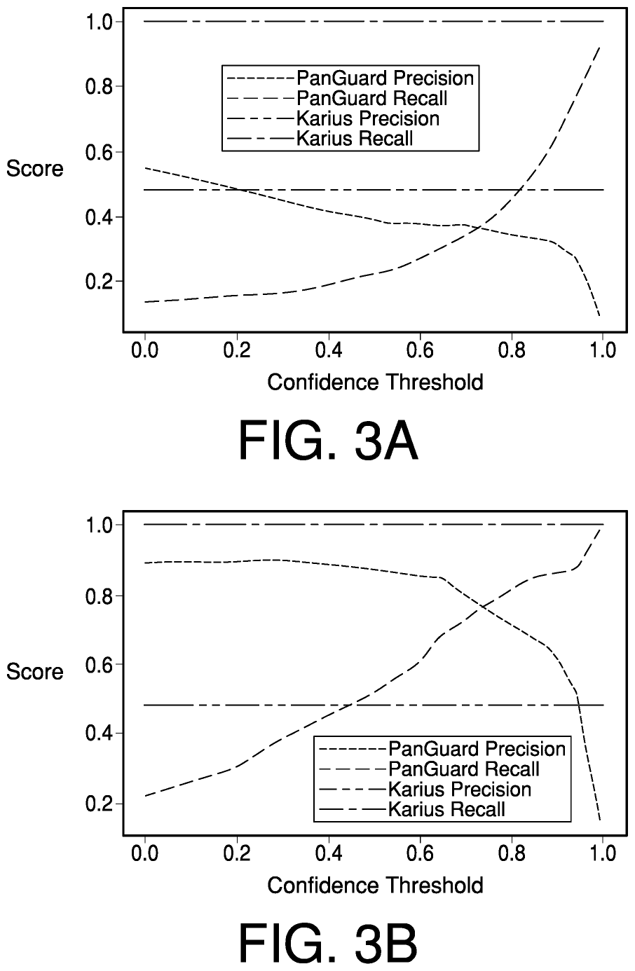

FIG. 3 shows a more detailed analysis of the PanGuard performance for copy numbers both above and below 50. These results show that PanGuard precision remains approximately 88% higher than the state of the art until copy numbers less than 50.

As shown in the figures, Battelle's SoC database results in high diagnostic precision in cases where the sample contains organisms for which SoCs are annotated and results in the added benefit of providing functional information to the end users (e.g., antibiotic resistance markers, toxins, etc.).

The recall in the case of Battelle's SoC database does suffer in a diagnostic setting due to organism coverage gaps. By using the 45 proteomes databases without Battelle's advanced informatic techniques results in performance comparable to that of state-of-the-art for identifying organisms in samples. By using the 45 proteomes database with Battelle's advance informatic techniques results in a 83% increase in performance over state-of-the-art for identifying organisms in samples. By using the ˜1,000 proteome database, PanGuard substantially still outperforms the state of the art by 45% higher performance with a 0.77 recall. For samples with agent copy number greater than or equal to 50, PanGuard has 88% higher performance than the state-of-the-art with a 0.72 recall.

However, PanGuard's performance decreases for multi-agent (˜10) with a low-copy number (<100) of agents in blood samples. Even in this case, PanGuard still predicts about 10% of the agents in this samples with 50% precision.

As provided in an exemplary embodiment, PanGuard's recall performance is dependent on the number of agents in the sample. As the number of agents in the sample increases, performance of PanGuard decreases. This decrease, along with low copy number samples, will likely only improve with greater sequencing depth. However, it is likely that in most clinically relevant samples analyzed that there will only one but at most a handful of clinically relevant agents present in the sample so the performance and recall of PanGuard will outperform existing technologies.

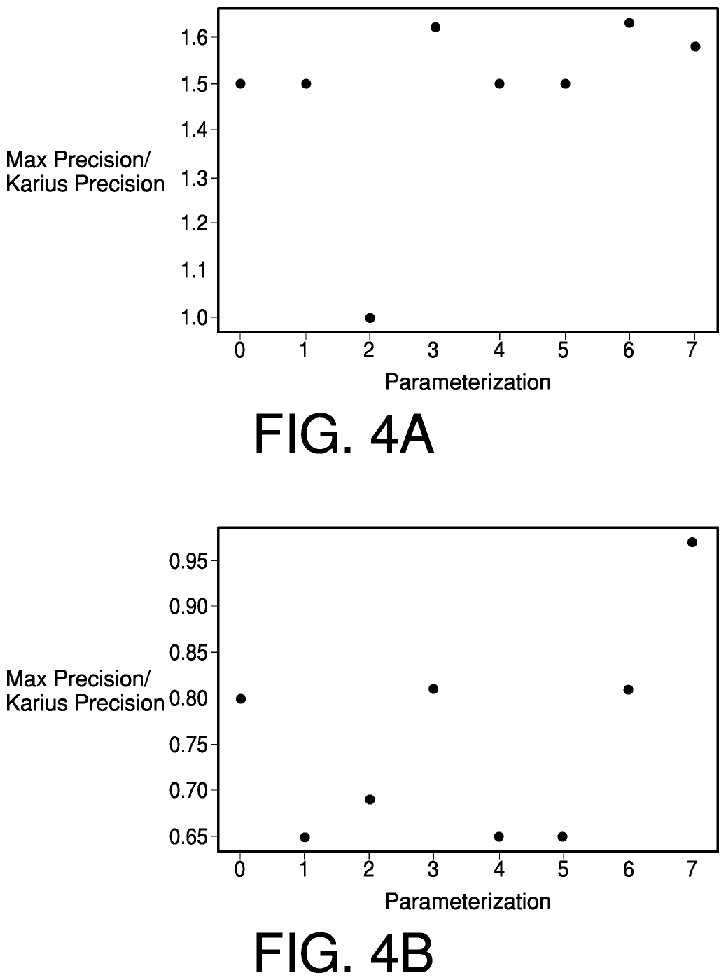

An exploration of the impact of parameterization on accuracy in predicting sample composition is shown in FIG. 4 for the following parameterizations:
n=m=p=1 (equally weighs agent uniqueness, proteome coverage, and sequence complexity)
n=m=1 & p=0 (equally weighs agent uniqueness and proteome coverage)
n=1 & m=p=0 (only considers proteome coverage) [Performed very poorly, due to protein variants and highly disparate agent concentrations in the sample]
n=p=0 & m=1 (only considers agent uniqueness)
n=1 & m=2 & p=0 (weighs agent uniqueness over proteome coverage)
n=2 & m=1 & p=0 (weighs proteome coverage over agent uniqueness)
p=1 & n=m=0 (only considers sequence complexity)
m=p=1 & n=0 (equally weighs agent uniqueness and sequence complexity) [Best Performing Parameterization]

### Exploration of the Impact of Aligner E-Value Cut Off on PanGuard Performance

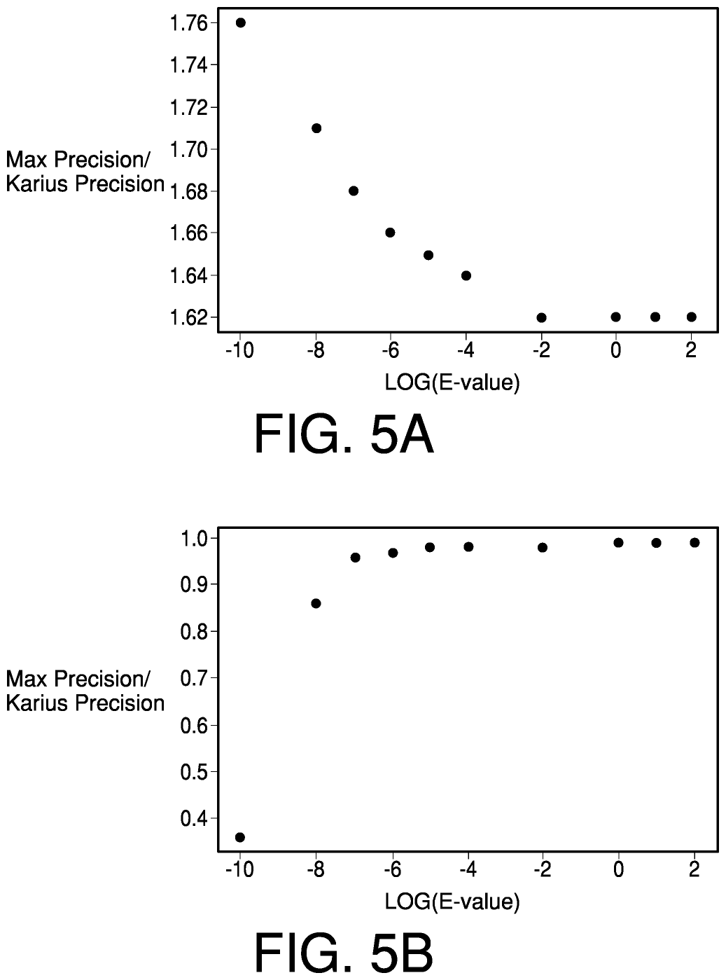

FIG. 5 shows the impact of aligner e-value cut off on PanGuard performance for the optimal parameterization found in the previous section. While precision monotonically increases as a function of decreasing e-value cut off, recall monotonically decreases as e-value decreases. Recall sharply decreases after an e-value cut off of 1E-7, however, setting 1E-7 to the optimum e-value cut off has the potential to result on poor performance on other datasets as it is right on the border of a dramatic reduction in recall. Thus, a safer range of e-value cut off's is from 1E-6 to 1E-4 (aligner default e-value cut off value). PanGuard's e-value cut off is conservatively set to 1E-4 to ensure recall is more likely to remain high. Additionally, after the fact pruning of results by e-value can be performed to optimize performance if a higher e-value cut off is selected. Note E-value is a function of database size, and this analysis will have to be reproduce anytime database size changes.

### ADDITIONAL ASPECTS

An additional exemplary case study is shown below that demonstrates the validity of the algorithm. In this case study, the taxonomic composition of a mock microbial community was determined from a from both long read (nanopore) and short read (Illumina) datasets as reported here:
https://academic.oup.com/gigascience/article/8/5/giz043/5486468.

For this case study, the same parameters were used as described above with the entire UniRef100 database as the reference database. The algorithm described here correctly predicted the taxonomic composition for all 12 species for both the long read and short read datasets.

| | Nanopore dataset: | Illumina dataset: |
|---|---|---|
| Species | Species detected? | species detected? |
| Bacillus subtilis | Yes | Yes |
| Listeria monocytogenes | Yes | Yes |
| Enterococcus faecalis | Yes | Yes |
| Staphylococcus aureus | Yes | Yes |
| Salmonella enterica | Yes | Yes |
| Escherichia coli | Yes | Yes |
| Pseudomonas aeruginosa | Yes | Yes |
| Lactobacillus fermentum | Yes | Yes |
| Saccharomyces cerevisiae | Yes | Yes |
| Cryptococcus neoformans | Yes | Yes |

### REFERENCES

[1] Jamison D T, Gelband H, Horton S, et al., Disease Control Priorities: Improving Health and Reducing Poverty. 3rd edition. 2017; Chapter 17
[2] https://www.cdc.gov/flu/weekly/overview.htm
[3] https://www.cdc.gov/surveillance/nrevss/index.html
[4] https://www.who.int/influenza/surveillance_monitoring/en/
[5] https://www.who.int/emergencies/diseases/en/
[6] Huang B, Jennison A, Whiley D, McMahon J, Hewitson G, Graham R, Jong A D, Warrilow D. Scientific Reports|(2019) 9:5409|https://doi.org/10.1038/s41598-019-41830-w
[7] Barbara Jester, Timothy Uyeki, Daniel Jernigan, Readiness for Responding to a Severe Pandemic 100 Years After 1918, American Journal of Epidemiology 2018; 187:12:2596-2602
[8] https://www.nature.com/articles/d41586-019-00277-9
[9] https://www.battelle.org/newsroom/press-releases/press-releases-detail/battelle-to-build-suite-of-mobile-analytical-labs-for-u.s.-department-of-defense
[10] https://globalbiodefense.com/2016/08/01/battelle-cbrne-defense-group-wins-two-major-rd-contracts/
[11] https://www.marketwatch.com/press-release/battelles-threatseqtm-service-wins-prestigious-rd-100-award-in-56th-annual-competition-2018-11-20
[12] https://www.technologyreview.com/2020/02/15/844752/biologists-rush-to-re-create-the-china-coronavirus-from-its-dna-code/
[13] Nicolas Janus, Launay-Vincent Vacher, Svetlana Karie, Elena Ledneva, Gilbert Deray, Vaccination and chronic kidney disease, Nephrology Dialysis Transplantation, Volume 23, Issue 3 (March 2008), Pages 800-807, https://doi.org/10.1093/ndt/gfm851
[14] Grumaz, S., Stevens, P., Grumaz, C. et al. Next-generation sequencing diagnostics of bacteremia in septic patients. Genome Med 8, 73 (2016). https://doi.org/10.1186/s13073-016-0326-8
[15] http://www.kariusdx.com and Blauwkamp, T. A., Thair, S., Rosen, M. J. et al. Analytical and clinical validation of a microbial cell-free DNA sequencing test for infectious disease. Nat Microbiol 4, 663-674 (2019). https://doi.org/10.1038/s41564-018-0349-6
[16] Atkinson, K., Bishop, L., Rhodes, G. et al. Nasopharyngeal metagenomic deep sequencing data, Lancaster, U K, 2014-2015. Sci Data 4, 170161 (2017). https://doi.org/10.1038/sdata.2017.161
[17] https://www.ncbi.nlm.nih.gov/genome/guide/human/
[18] Hannes Hauswedell, Jochen Singer, Knut Reinert, Lambda: the local aligner for massive biological data, Bioinformatics, Volume 30, Issue 17, 1 Sep. 2014, Pages i349-i355, https://doi.org/10.1093/bioinformatics/btu439
[19] Graf E H, Simmon K E, Tardif K D, Hymas W, Flygar S, Eilbeck K, Yandell M, Schlaberg R. 2016. Unbiased Detection of Respiratory Viruses by Use of RNA Sequencing-Based Metagenomics: a Systematic Comparison to a Commercial PCR Panel. Journal of Clinical Microbiology. 54(4): 1000-1007.
[20] Fischer et al. 2015. Evaluation of Unbiased Next-Generation Sequencing of RNA (RNA-seq) as a Diagnostic Method in Influenza Virus-Positive Respiratory Samples. Journal of Clinical Microbiology. 53(7): 2238-2250.
[21] https://nextgendiagnostics.ucsf.edu/wp-content/uploads/2019/04/Miller_Genome-Res-2019.pdf
[22] https://www.nature.com/articles/s41576-019-0113-7

1. A method to identify or to predict a pathological agent, comprising:
a) receiving a dataset of metagenomes from uninfected people or nonhuman animals;
b) receiving an infected dataset;
c) developing a database of clinically relevant proteomes by processing the dataset of metagenomes and the infected dataset;
d) aligning datasets using an aligner tool;
e) removing alignments with less than 99% identity and an alignment length of less than 48 bps;
f) scoring the retained alignments;
g) calculating an agent confidence value from the scoring;
h) predicting an agent based on a confidence threshold; and
i) calculating a performance metric relative to data from a pre-existing method of predicting a disease outbreak.

2. The method of claim 1 wherein the infected dataset in step b) is developed by converting FastQ and fna datasets into Fasta files.

3. The method of claim 1 wherein the aligner tool is a lambda aligner.

4. The method of claim 1, wherein the scoring in step d) is S_{a,r,acc} produced from:
$${S}_{a , r , acc} = \frac{{Rcov}_{acc , r} \times {Nuacc}_{a}^{n}}{{Na}_{r}^{m} \times {Nacc}_{r}^{p}} ,$$
where Rcov_{acc,r} is a measure alignment quality of the query subsequence associated with region r to subject accession acc, Nuaccₐⁿ is the number of unique accession associated with an agent found across the entire metagenomic sample, Naᵣ is the number of unique agents associated with region r, Naccᵣ is the number of accessions associated with region r, and m, n, and p belong to the integers.

5. The method as recited in claim 1, wherein the scoring in step g) is S_{a,r,acc} produced from:
$${S}_{a , r , acc} = \frac{{Rcov}_{acc , r}}{{Na}_{r} \times {Nacc}_{r}}$$
wherein Rcov_{acc,r} is a measure alignment quality of the query subsequence associated with region r to subject accession acc, Naᵣ is the number of unique agents associated with the subject accessions in the region r, and Naccᵣ is the number of accessions from the subject database that associates with the region.

6. The method of claim 1, wherein the agent confidence value in step g) is produced from:
a) obtaining an agent region score, S_{a,r}, which is calculated to be the score associated with the highest scoring accession for the given agent, region combination:
$${S}_{a , r} = {max}_{acc} {S}_{a , r , acc} ;$$
b) obtaining an agent score, Sₐ, which is calculated as follows:
$${S}_{a} = {\sum }_{r} {S}_{a , r} ;$$
and
c) obtaining an agent confidence, Cₐ, which is defined as:
$${C}_{a} = \frac{{S}_{a}}{{\sum }_{a} {S}_{a}} .$$

7. The method of claim 6, wherein the agent confidence value in step g) is further obtained by an iterative process comprising:
a) sorting the agents from highest to lowest agent confidence;
b) pulling all regions that are associated with the highest confidence agent;
c) setting the agent, region score equal to zero for all other agents in these regions;
d) recalculating agent confidences;
e) pulling all regions associated with the next highest confidence agent;
f) setting the agent, region score equal to zero for all other agents in these regions; and
g) recalculating agent confidences.
wherein steps e)-g) are repeated until all remaining agents have been iterated over.

8. The method of claim 1, wherein the confidence threshold is produced based on a training data or wherein an optional K-means clustering of the agent scores by domain can be calculated and the final taxonomies are those associated with the top cluster within each domain (within a threshold).

9. The method as recited in claim 1, wherein the performance metric in step h) is calculated as precision and recall from:
$$Precision = \frac{TP}{TP + FP} and Recall = \frac{TP}{TP + FN} .$$
such that TP=true positive, TN=true negative, FP=false positive, and FN=false negative; and TP, TN, FP and FN are calculated by comparing the set of predicted agents from step 8) to a set of verified agents from published literatures.

10. A method of detecting a disease outbreak, comprising:
a) establishing a cohort of individuals for repeated collection of samples;
b) repeatedly collecting samples from the individuals in the cohort;
c) analyzing the samples to identify at least one pathological agent according to claim 1; and
d) determining whether the identified at least one pathological agent indicate a disease outbreak.

11. The method of claim 10, wherein the cohort of individuals comprises consenting patients such as front line workers, dialysis patients, other immunocompromised patients, or another selected cohort.

12. The method of claim 10, wherein step b) further comprises sequencing the samples using high throughput screening.

13. The method as recited in claim 1, wherein the pathological agent is identified in view of a curated data collection comprises at least 1600 proteomes from clinically relevant pathological agents.

14. The method of claim 10, further comprising transmitting an alert indicating the disease outbreak.

15. A method for making a taxonomic composition prediction, comprising:
a) an alignment step against a subject database of protein and/or nucleotide sequences;
b) a filtering step comprising removing low quality alignments;
c) scoring step comprising:
scoring sequence level taxonomy predictions based on an information content; combining sequence-level taxonomy prediction scores into sample-level taxonomy scores;
d) a finding step comprising finding all reads associated with the highest scoring taxonomy that has not yet been processed and removing all other taxonomies and associated accession from the reads; and
wherein the scoring step results in one or more sample-level taxonomy scores; and conducting a K-means cluster of the sample-level taxonomy scores by domain and set the taxonomies associated with a top cluster to a final taxonomic composition prediction.

16. The method of claim 15, wherein step b) includes anomalous reads.

17. The method of claim 15, wherein the sequence-level taxonomy prediction scoring in step c) is achieved by S_{a,r,acc} produced from:
$${S}_{a , r , acc} = \frac{{Rcov}_{acc , r} \times {Nuacc}_{a}^{n}}{{Na}_{r}^{m} \times {{N}_{acc}}_{r}^{p}} ,$$
where Rcov_{acc,r} is a measure alignment quality of the query subsequence associated with region r to subject accession acc, Nuaccₐⁿ is the number of unique accession associated with an agent found across the entire metagenomic sample, Naᵣ is the number of unique agents associated with region r, Naccᵣ is the number of accessions associated with region r, and m, n, and p belong to the integers.

18. The method as recited in claim 15, wherein the sequence-level taxonomy prediction scoring in step c) is achieved by S_{a,r,acc} produced from:
$${S}_{a , r , acc} = \frac{{Rcov}_{acc , r}}{{Na}_{r} \times {Nacc}_{r}}$$
wherein Rcov_{acc,r} is a measure alignment quality of the query subsequence associated with region r to subject accession acc, Naᵣ is the number of unique agents associated with the subject accessions in the region r, and Naccᵣ is the number of accessions from the subject database that associates with the region.

19. The method of claim 15, wherein in step c) the sample-level taxonomy scores are achieved by:
a) obtaining a taxonomic composition region score, S_{a,r}, which is calculated to be the score associated with the highest scoring accession for the given taxonomic composition, region combination:
$${S}_{a , r} = {max}_{acc} {S}_{a , r , acc} ;$$
b) obtaining sample-level taxonomy score, Sₐ, which is calculated as follows:
$${S}_{a} = {\sum }_{r} {S}_{a , r}$$

20. The method as recited by claim 15 wherein step 4) further includes iteratively repeating scoring until all taxonomies have been processed.

21-25. (canceled)
